(19) United States
(12) Patent Application Publication
(10) Pub. No.: US 2023/0285742 A1
(43) Pub. Date: Sep. 14, 2023

(54) SYSTEMS AND METHODS FOR IN-BODY SECURITY EMPLOYING HARDWARE-LEVEL SYSTEMS IN BIDIRECTIONAL NEURAL INTERFACES

(71) Applicant: PRECISION NEUROSCIENCE CORPORATION, New York, NY (US)

(72) Inventors: Benjamin I. RAPOPORT, New York, NY (US); Manuel MONGE, New York, NY (US)

(73) Assignee: PRECISION NEUROSCIENCE CORPORATION, New York, NY (US)

(21) Appl. No.: 18/180,248

(22) Filed: Mar. 8, 2023

Related U.S. Application Data
(60) Provisional application No. 63/317,706, filed on Mar. 8, 2022.

(51) Int. Cl. A61N 1/04

(52) U.S. Cl. CPC A61N 1/04; A61N 1/0526

(57) ABSTRACT

Disclosed are systems and methods for circuit- and system-level techniques and approaches for data encryption in scalable, high-bandwidth, bidirectional neural interfaces. In some embodiments, the disclosed systems and methods may be configured to encrypt neural data as close as possible to the data source, and before the data is transmitted out of the body. In some embodiments, the disclosed systems and methods may utilize hardware-based approaches, including those involving modified analog-to-digital converters, and symmetric cryptographic algorithms.

### PRIORITY

The application claims priority to U.S. Provisional Patent Application No. 63/317,706, titled SYSTEMS AND METHODS FOR IN-BODY SECURITY EMPLOYING HARDWARE-LEVEL SYSTEMS IN BIDIRECTIONAL NEURAL INTERFACES, filed Mar. 8, 2022, which is hereby incorporated by reference herein in its entirety.

### BACKGROUND

Neural interfaces are biomedical devices that may be used to study and investigate the nervous system and the brain, and to develop diagnosis and treatment options for brain-related disorders. Applications for neural interfaces include, but are not limited to, motor prostheses, deep-brain stimulation, visual and hearing prostheses, brain-function mapping, seizure detection, and the like. Neural interfaces may be used for several applications including diagnostics, therapeutics, basic research and brain-machine or brain-computer interfaces.

Neural interfaces may involve the capture of data generated by the brain of a patient or user. This data may be viewed as intimately private and deserving of extreme security. Conventional neural interfaces may produce unsecured neural data that freely generates electromagnetic signatures that could compromise the privacy, security, and safety of the patient or user.

As neural interfaces mature, it is extremely important to address the security needs of these neural interface devices and the data they produce. Conventional methods for addressing the security concerns associated with neural interfaces have involved solutions similar to the approaches taken with regards to low-power internet-of-things (IoT) devices. However, conventional systems have not developed hardware-level solutions to address all of the security concerns associated with neural interfaces.

### SUMMARY

Disclosed herein are systems and methods for circuit- and system-level techniques and approaches for data encryption in scalable, high-bandwidth, bidirectional neural interfaces. In some embodiments, the disclosed systems and methods may be configured to encrypt neural data as close as possible to the data source, and before the data is transmitted out of the body. In some embodiments, the disclosed systems and methods may utilize hardware-based approaches.

Systems and methods such as those disclosed herein will be important to ensure the privacy and security of neural data for patients and other users of neural interfaces as these devices are employed in a variety of applications.

In some embodiments a neural interface device includes an electrode array configured to stimulate or record from neural tissue adjacent to the electrode array, and an integrated circuit in electrical communication with the electrode array, the integrated circuit having an analog-to-digital converter (ADC) producing digitized electrical signal output, where the ADC has an encryption module, where the encryption module encrypts the digitized electrical signal output of the ADC. Optionally, the ADC includes a successive approximation register (SAR) architecture. Optionally, the encryption module includes a bit stream cipher, where the encryption module applies the bit stream cipher to the digitized electrical signal output of the ADC. Optionally, the encryption module includes a block stream cipher, where the encryption module applies the block stream cipher to the digitized electrical signal output of the ADC. Optionally, a cipher of the encryption module takes as input 1-, 8-, 128-, 192- or 256-bits. The neural interface device may also include a wireless transmitter communicatively coupled to the integrated circuit or the encryption module and an external processor. The neural interface device may also include control logic for operating the integrated circuit or electrode array, memory for storing recordings from the electrode array, and a power management unit for providing power to the integrated circuit or electrode array.

In some embodiments a neural interface device includes an electrode array configured to stimulate or record from neural tissue adjacent the electrode array, and an integrated circuit in electrical communication with the electrode array, the integrated circuit having a successive approximation register (SAR) analog-to-digital converter (ADC), where the SAR ADC includes a cipher module, where the cipher module applies a cipher to an electrical signal output by a digital-to-analog circuit (DAC) of the SAR ADC to generate an encrypted serial output. Optionally, the cipher could include bit stream cipher or a block cipher. Optionally, the SAR ADC includes a sample and hold circuit configured to receive the electrical signal from the electrode array, a comparator electrically coupled to the sample and hold circuit, where the comparator compares the received electrical signal to a reference signal of the DAC, where the DAC is electrically coupled to the comparator, and a binary search algorithm electrically coupled to the comparator, where the binary search algorithm generates a digital electrical signal representative of the received electrical signal. Optionally, the cipher module takes as input at least one of 1-, 8-, 128-, 192-, or 256-bits. Optionally, the neural interface device may include a wireless transmitter communicatively coupled to the integrated circuit or a cipher block module, and an external processor. In some embodiments, the neural interface device includes control logic for operating the integrated circuit or electrode array, memory for storing recordings from the electrode array, and a power management unit for providing power to the integrated circuit or electrode array.

In some embodiments, a neural interface device includes an electrode array configured to stimulate or record from neural tissue adjacent the electrode array, at least one recording array, each of the at least one recording array having at least one integrated circuit in electrical communication with the electrode array, where each of the at least one integrated circuits includes a successive approximation register (SAR) analog-to-digital converter (ADC), where the SAR ADC receives an electrical signal from the at least one recording array and outputs a digital electrical signal, and a stream cipher module can that implement a symmetric cryptographic algorithm, where the stream cipher module applies the symmetric cryptographic algorithm to the digital electrical signal output by the SAR ADC to generate serial encrypted output. Optionally, the SAR ADC includes a sample and hold circuit configured to receive the electrical signal from the at least one recording array, a comparator electrically coupled to the sample and hold circuit, where the comparator compares the received electrical signal to a reference signal of a digital-to-analog circuit (DAC) of the SAR ADC, where the DAC is electrically coupled to the comparator, and a binary search algorithm electrically coupled to the comparator, where the binary search algorithm generates the digital electrical signal representative of the received electrical signal.

In some embodiments, a neural interface device includes an electrode array configured to stimulate or record from neural tissue adjacent the electrode array, a plurality of recording arrays each having at least one integrated circuit in electrical communication with the electrode array, where each of the at least one integrated circuits includes a successive approximation register (SAR) analog-to-digital converter (ADC), where the SAR ADC receives an electrical signal from the plurality of recording arrays and outputs a digital electrical signal, and a block cipher module that can implement a symmetric cryptographic algorithm, where the block cipher module applies the symmetric cryptographic algorithm to the digital electrical signal output by the SAR ADC to generate serial encrypted output. Optionally, the SAR ADC includes a sample and hold circuit configured to receive the electrical signal from the at least one recording array, a comparator electrically coupled to the sample and hold circuit, where the comparator compares the received electrical signal to a reference signal of a digital-to-analog circuit (DAC) of the SAR ADC, where the DAC is electrically coupled to the comparator, and a binary search algorithm electrically coupled to the comparator, where the binary search algorithm generates the digital electrical signal representative of the received electrical signal.

In some embodiments, a neural interface device includes an electrode array configured to stimulate or record from neural tissue adjacent the electrode array, a plurality of recording arrays each having at least one integrated circuit in electrical communication with the electrode array, where each of the at least one integrated circuits includes a successive approximation register (SAR) analog-to-digital converter (ADC), where the SAR ADC receives an electrical signal from the plurality of recording arrays and outputs a digital electrical signal, and an unrolled pipelined cipher module that can implment a symmetric cryptographic algorithm, where the unrolled pipelined cipher module applies the symmetric cryptographic algorithm to the digital electrical signal output by the SAR ADC to generate serial encrypted output. Optionally, the SAR ADC includes a sample and hold circuit configured to receive the electrical signal from at least one recording array, a comparator electrically coupled to the sample and hold circuit, where the comparator compares the received electrical signal to a reference signal of a digital-to-analog circuit (DAC) of the SAR ADC, where the DAC is electrically coupled to the comparator, and a binary search algorithm electrically coupled to the comparator, where the binary search algorithm generates the digital electrical signal representative of the received electrical signal.

In some embodiments, a neural interface device includes an electrode array configured to stimulate or record from neural tissue adjacent the electrode array, and an integrated circuit in electrical communication with the electrode array, the integrated circuit including a successive approximation register (SAR) analog-to-digital converter (ADC), where the SAR ADC receives an electrical signal from the plurality of recording arrays and outputs a digital electrical signal passed through an accumulator of the SAR ADC, and a bit stream cipher module, where the bit stream cipher module applies a bit stream cipher to the digital electrical signal output by the accumulator of the SAR ADC to generate an encrypted serial output. The SAR ADC may include a sample and hold circuit configured to receive the electrical signal from at least one recording array, a multiplexor and the accumulator electrically coupled to the sample and hold circuit, where the accumulator gathers electrical signals, a comparator electrically coupled to the accumulator, where the comparator compares the received electrical signal to a reference signal of a digital-to-analog circuit (DAC) of the SAR ADC, where the DAC is electrically coupled to the comparator, and a binary search algorithm electrically coupled to the comparator, where the binary search algorithm generates the digital electrical signal representative of the received electrical signal. The bit stream cipher module may take as input at least one of 1-, 8-, 128-, 192-, or 256-bits. The neural interface device may also include a wireless transmitter communicatively coupled to the integrated circuit or a cypher block module, and an external processor. The neural interface device may also include control logic for operating the integrated circuit or electrode array, memory for storing recordings from the electrode array, and a power management unit for providing power to the integrated circuit or electrode array.

In some embodiments, a neural interface device may include an electrode array configured to stimulate or record from neural tissue adjacent the electrode array, a plurality of recording arrays each having at least one integrated circuit in electrical communication with the electrode array, where each of the at least one integrated circuits includes a successive approximation register (SAR) analog-to-digital converter (ADC), where the SAR ADC receives an electrical signal from the plurality of recording arrays and outputs a digital electrical signal passed through an accumulator of the SAR ADC, and a stream cipher module, where the stream cipher module applies a byte stream cipher to the digital electrical signal output by the accumulator of the SAR ADC to generate an encrypted serial output.

In some embodiments, a neural interface device may include an electrode array configured to stimulate or record from neural tissue adjacent the electrode array, at least one recording array, each of the at least one recording array having at least one integrated circuit in electrical communication with the electrode array, where each of the at least one integrated circuits includes a successive approximation register (SAR) analog-to-digital converter (ADC), where the SAR ADC includes a sample and hold circuit, a multiplexor and an accumulator positioned after the sample and hold circuit, a comparator, a digital-to-analog circuit (DAC), a switch array, and a binary search algorithm, and a block cipher module, where the block cipher module applies a block cipher to an electrical signal output by the accumulator of the SAR ADC to generate an encrypted serial output. Optionally, the neural interface device may have a sample and hold circuit configured to receive the electrical signal from at least one recording array, a multiplexor and the accumulator electrically coupled to the sample and hold circuit, where the accumulator gathers electrical signals, a comparator electrically coupled to the accumulator, where the comparator compares the received electrical signal to a reference signal of a digital-to-analog circuit (DAC) of the SAR ADC, where the DAC is electrically coupled to the comparator, and a binary search algorithm electrically coupled to the comparator, where the binary search algorithm generates the digital electrical signal representative of the received electrical signal.

In some embodiments, a neural interface device includes an electrode array configured to stimulate or record from neural tissue adjacent the electrode array, at least one recording array, each of the at least one recording array having at least one integrated circuit in electrical communication with the electrode array, where each of the at least one integrated circuits includes a successive approximation register (SAR) analog-to-digital converter (ADC), where the SAR ADC receives an electrical signal from the plurality of recording arrays and outputs a digital electrical signal passed through an accumulator of the SAR ADC, and an unrolled pipelined cipher module, where the unrolled pipelined cipher module applies an unrolled pipelined cipher to the digital electrical signal output by the accumulator of the SAR ADC to generate an encrypted serial output. Optionally, the SAR ADC may include a sample and hold circuit configured to receive the electrical signal from the at least one recording array, a multiplexor and the accumulator electrically coupled to the sample and hold circuit, where the accumulator gathers electrical signals, a comparator electrically coupled to the accumulator, where the comparator compares the received electrical signal to a reference signal of a digital-to-analog circuit (DAC) of the SAR ADC, where the DAC is electrically coupled to the comparator, and a binary search algorithm electrically coupled to the comparator, where the binary search algorithm generates the digital electrical signal representative of the received electrical signal.

In some embodiments, a neural interface device includes an electrode array configured to record from neural tissue adjacent the electrode array, at least one recording array, each of the at least one recording array having at least one integrated circuit in electrical communication with the electrode array, one or more processing blocks in communication with the at least one recording array and configured to extract features from the electrical signals received by the at least one recording array, and an encryption module that can implement a symmetric cryptographic algorithm, where the encryption module applies a symmetric cryptographic algorithm to the electrical signals after the one or more processing blocks has extracted features from the electrical signals.

### BRIEF DESCRIPTION

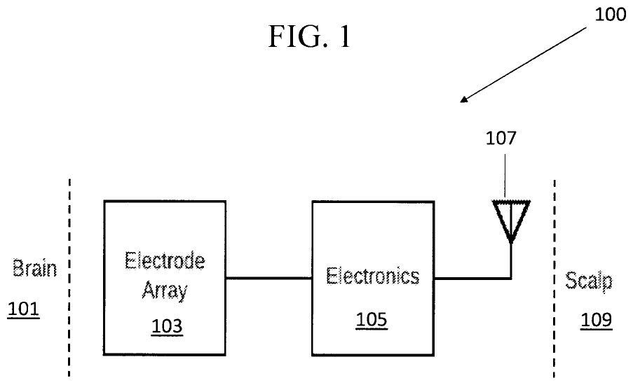

FIG. 1 illustrates a neural interface in accordance with some embodiments of the present disclosure.

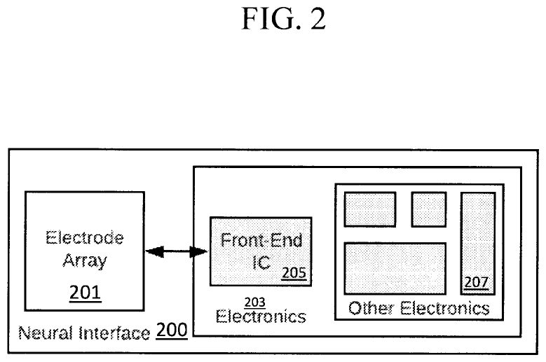

FIG. 2 illustrates hardware level components of a neural interface in accordance with some embodiments of the present disclosure.

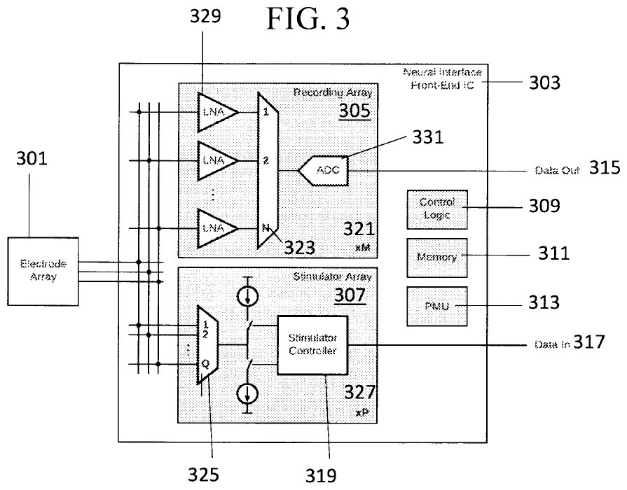

FIG. 3 illustrates an integrated circuit for a neural interface in accordance with some embodiments of the present disclosure.

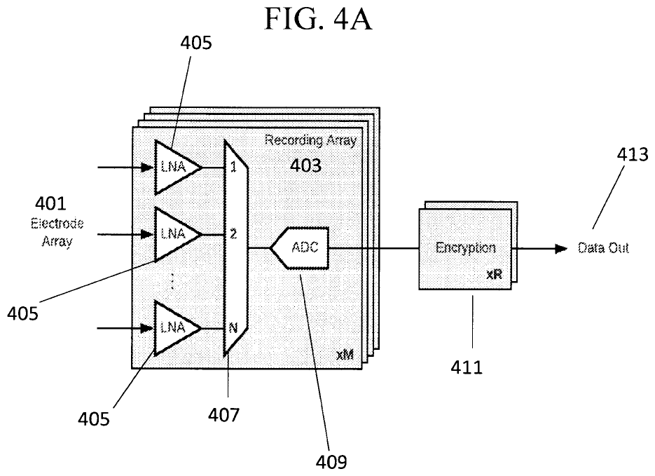

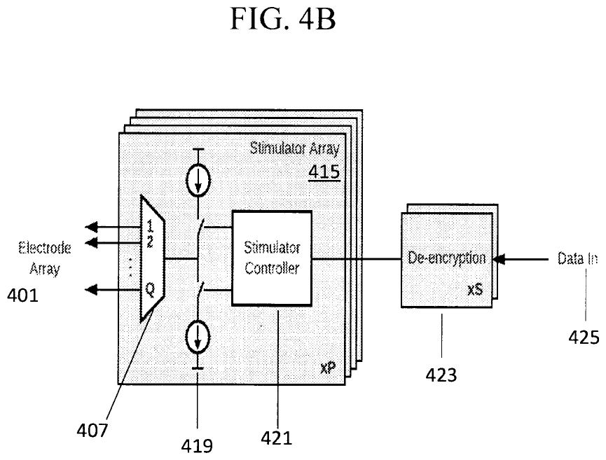

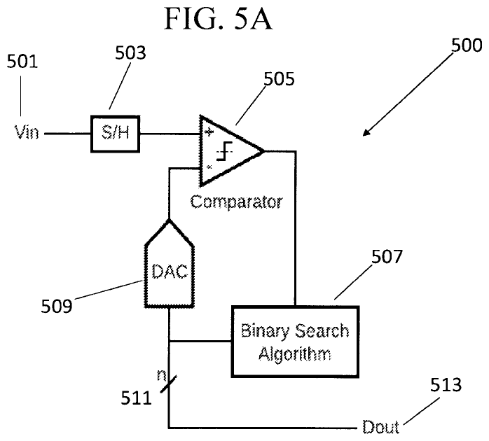

FIG. 4A illustrates a recording array for a neural interface in accordance with some embodiments of the present disclosure.

FIG. 4B illustrates a stimulator array for a neural interface in accordance with some embodiments of the present disclosure.

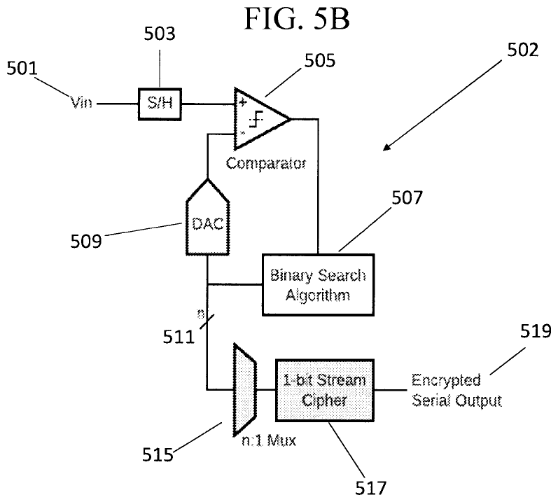

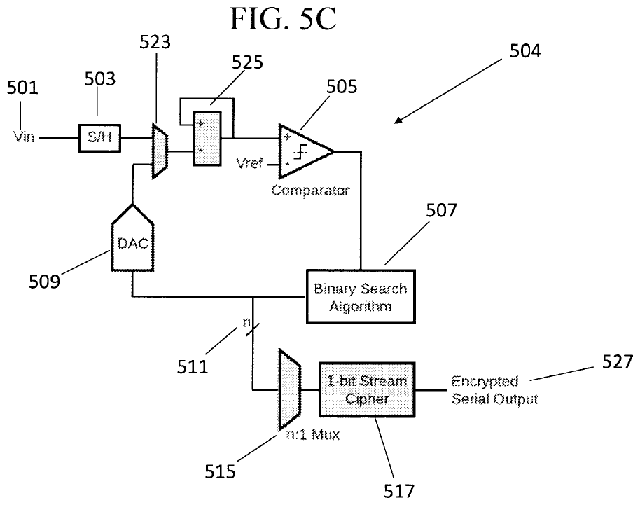

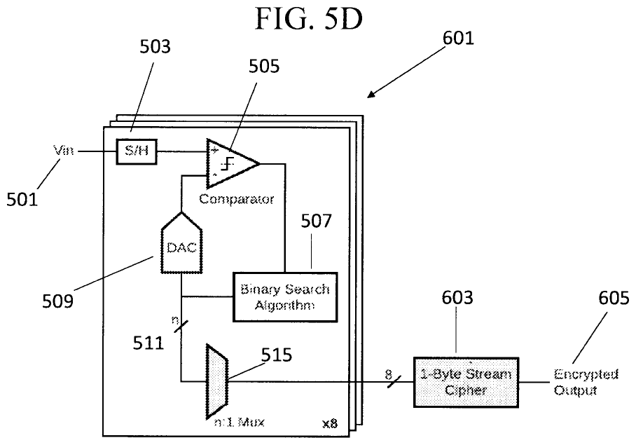

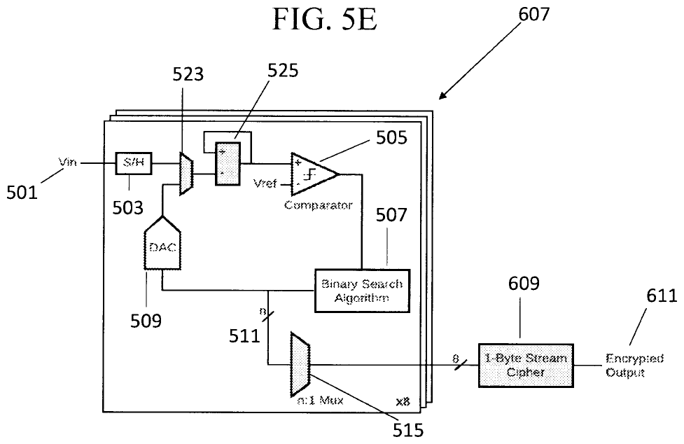

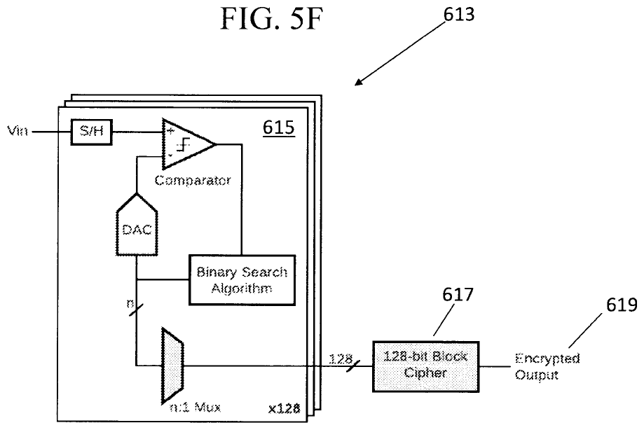

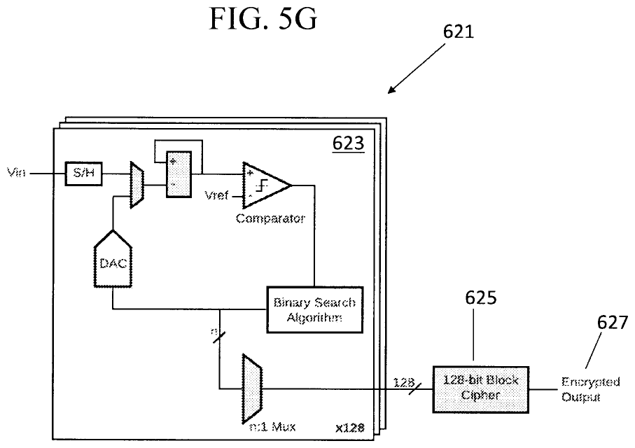

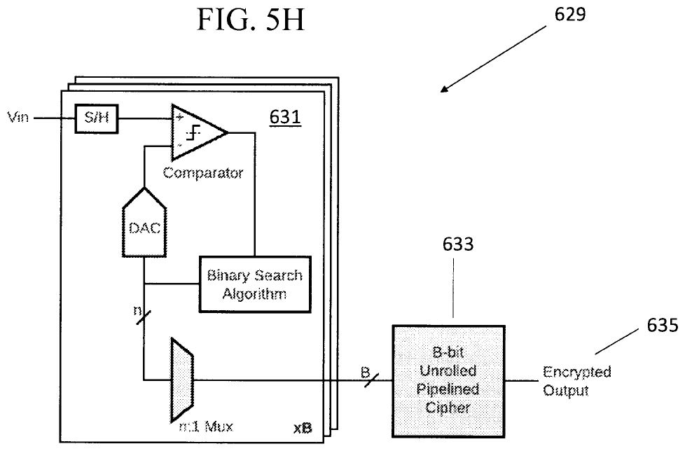

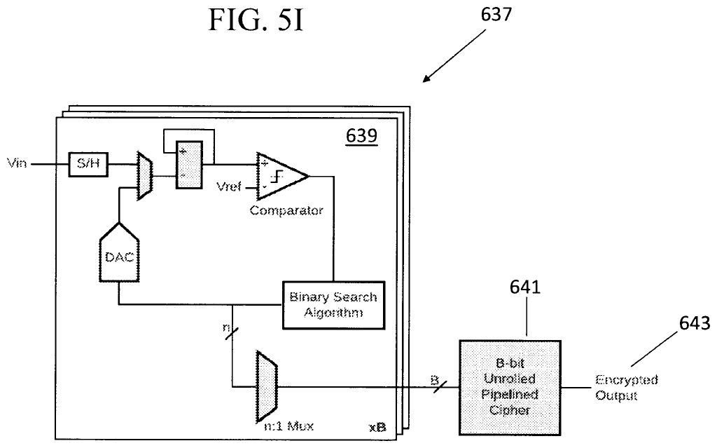

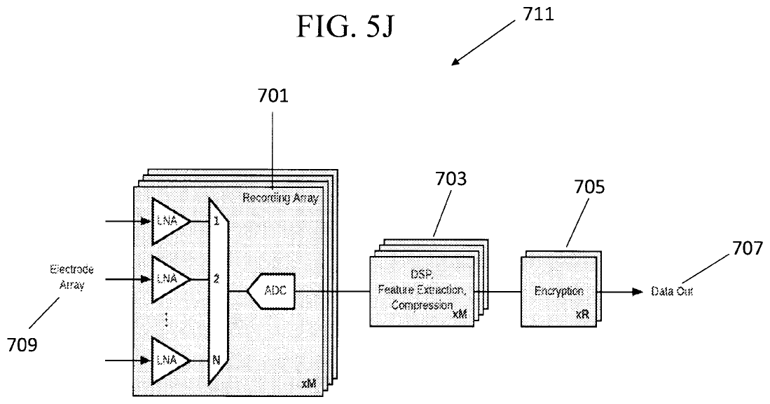

FIG. 5A illustrates a circuit block diagram for a successive-approximation register (SAR) analog to digital converter (ADC) in accordance with some embodiments of the present disclosure.

FIG. 5B illustrates a circuit block diagram for a modified analog to digital converter (ADC) in accordance with some embodiments of the present disclosure.

FIG. 5C illustrates a circuit block diagram for a modified analog to digital converter (ADC) in accordance with some embodiments of the present disclosure.

FIG. 5D illustrates a circuit block diagram for a plurality of modified analog to digital converter (ADC) in accordance with some embodiments of the present disclosure.

FIG. 5E illustrates a circuit block diagram for a plurality of modified analog to digital converters (ADCs) in accordance with some embodiments of the present disclosure.

FIG. 5F illustrates a circuit block diagram for a plurality of modified analog to digital converters (ADCs) in accordance with some embodiments of the present disclosure.

FIG. 5G illustrates a circuit block diagram for a plurality of modified analog to digital converters (ADCs) in accordance with some embodiments of the present disclosure.

FIG. 5H illustrates a circuit block diagram for a plurality of modified analog to digital converters (ADCs) in accordance with some embodiments of the present disclosure.

FIG. 5I illustrates a circuit block diagram for a plurality of modified analog to digital converters (ADCs) in accordance with some embodiments of the present disclosure.

FIG. 5J illustrates a circuit block diagram for an implementation of encryption in accordance with some embodiments of the present disclosure.

### DETAILED DESCRIPTION

FIG. 1 illustrates an implant 100 in connection with a neural interface. In some embodiments, the implant 100 could include a brain implant, such as is shown in FIG. 1. In other embodiments, the implant 100 could include other electronic devices that are implantable through the body (e.g., in the chest cavity). In some embodiments, the neural interface may be a biomedical device configured to study, investigate, diagnose, treat, and/or augment brain activity. In some embodiments, the implant 100, may be positioned between the brain 101 and the scalp 109. The implant 100 may include an electrode array 103 configured to record and/or stimulate an area of the brain 101. The electrode array 103 may be connected to an electronics hub 105 which is configured to transmit via wireless or wired transmitter 107 to an external device. In some embodiments, the external device may be a tablet, smartphone, laptop, desktop, secure server, smartwatches, head-mounted virtual and/or augmented reality devices, smart inductive charger devices, or any other suitable device.

In conventional systems without security features, the brain implant could be accessed by malicious actors to cause perturbations or modifications to the nervous systems, thus posing additional security risks for the user of a brain implant. For example, if the brain implant was used for recording, the amplification and digitization of neural signals would increase the power of the electromagnetic (EM) fields they produce and could cause security issues when, for instance, these digitized signals are transmitted via a wire or wirelessly. In another example, if the brain implant was used for stimulation, interception, modification or replacement of stimulating commands may cause significant harm to the patient or user. Further, even at baseline recording or stimulation, neural devices may be vulnerable to security issues.

In some embodiments, the present disclosure provides hardware-based solutions to preserving the privacy and security of data recorded and transmitted by neural interfaces (e.g., brain-machine interfaces, brain-computer interfaces, brain implants). The disclosed systems and methods provide hardware-level security where the data is generated (such as for neural interfaces that record) or used (such as for neural interfaces that stimulate) in order to enable the use of neural interfaces for practical and clinical applications.

In some embodiments, to increase security, the disclosed systems and methods provide for end-to-end encryption of recording signals and stimulating commands between the origin and destination of the data, and at-rest if data is stored in a different location (e.g., secured servers). In some embodiments, the disclosed system may encrypt data everywhere it is at-rest, including the origin device, which may provide protections against the device being compromised. In some embodiments, the disclosed systems may encrypt data as close as possible to digitization, such that non-encrypted bits are not generated, or encrypted shortly after generation.

FIG. 2 illustrates hardware level systems for in-body security in bi-directional neural interfaces (capable of one or more of recording and stimulating). In particular, FIG. 2 illustrates an integrated circuit (IC) that is configured to be used within a neural interface. The IC may include one or more chips that control and perform the recording and/or stimulation of neural tissue. In some embodiments, hardware-level security may be achieved by enabling the encryption of all data and commands sent to and from the chip.

As illustrated in FIG. 2, a neural interface 200 may include an electrode array 201 configured to stimulate and/or record from neural tissue. The electrode array 201 may be coupled to an electronics module 203 including front-end integrated circuits 205 and other electronics components 207 such as those required to power, record, stimulate, and the like.

Conventional neural devices have thus far been unable to achieve hardware-level security because as the number of channels interfacing with the brain continues to increase from few tens or hundreds to more than a few thousands, the size and power of the ICs become critical because of the safety limitations on power dissipation near the brain and neural tissue, and available surgical techniques. The disclosed systems and methods address this problem through the use of circuit-level techniques and approaches for data encryption in scalable, high-bandwidth, bidirectional neural interfaces. For example, the disclosed systems and methods may perform circuit-level techniques that can enable the low-power and compact implementation of hardware-friendly encryption algorithms such as the advanced encryption standard (AES), Sa1sa20, and the like.

FIG. 3 illustrates the system architecture of a general high-bandwidth neural interface front-end integrated circuit, such as the front-end IC 205 of FIG. 2. As illustrated in FIG. 3, the neural interface front-end IC 303 may be connected to an electrode array. The neural interface front-end IC may also include a recording array 305 and a stimulator array 307, each of which is configured to record from or stimulate neural tissue, respectively, using the electrode array 301.

The recording array may produce data 315 corresponding to recorded neural activity. In some embodiments, the stimulator array may receive stimulation control data 317 corresponding to signals for controlling operation of the stimulation provided by the electrode array 301. The received stimulation control data 317 may be routed to a stimulator controller 319 within the stimulator array 307. Further, the neural interface front-end IC 303 may include control logic 309, memory 311, and a power management unit (PMU) 313.

As illustrated in FIG. 3, the number of channels associated with the neural interface front-end IC 303 may reach several thousands. For example, the neural interface front-end IC 303 may include a plurality of recording arrays (M) 321 each of which is configured to record from N number 323 of channels. Similarly, the neural interface front-end IC 303 may include a plurality of stimulator arrays (P) 327 each of which is configured to be multiplexed across Q channels 325.

The chip-level system architecture of a general high-bandwidth neural interface front-end IC illustrated in FIG. 3 demonstrates the key characteristics that make them a challenging problem for the implementation of cryptographic algorithms.

As illustrated in FIG. 3, high bandwidth neural interfaces may include thousands of electrodes. Typically, a single low-noise amplifier (LNA) 329 is used per channel, and a group of LNAs (e.g., 8-16 low-noise amplifiers) share a single analog to digital converter (ADC) 331. Thus, scalable, high-bandwidth neural recording arrays typically have hundreds of ADCs that run in parallel. The disclosed systems allowed for shared resources, thus providing power efficiencies while at the same time providing for functionality. The amount of data produced by a recording array such as recording array 305 is immense and with a data rate on the order of many gigabits per second. For example, assuming an ADC resolution for neural signals of 10 bits, the amount of data produced is in the order of Gb/s, with multiple bits in parallel. This characteristic of generating such high amount of data in parallel bits is unique to scalable, high-bandwidth neural interfaces. However, the challenge of processing such high amounts of data is exacerbated by the low-power and small-area requirements of neural interfaces which limit the amount of computation that can be done in the neural interface front-end IC 303 since the power dissipated by the neural interface front-end IC 303 should not increase the temperature of the brain or neural tissue by more than 1° C. or 2° C. Accordingly, some cryptographic algorithms such as asymmetric cryptographic algorithms, may require higher power and processing capabilities are inappropriate for neural interfaces as they may dangerously increase the temperature of the associated brain or neural tissue.

By contrast, the disclosed embodiments may provide for systems and methods for symmetric cryptographic algorithms that may be implemented in scalable, high-bandwidth neural interfaces due to their lower complexity and better hardware compatibility than asymmetric cryptographic algorithms. Examples of such symmetric cryptographic algorithms include both stream and block ciphers such as Salsa20 and AES-128.

In some embodiments, symmetric, hardware-friendly encryption algorithms can be directly integrated into the system. For example, as illustrated in FIG. 4A, a recording array 403 may record signals via an electrode array 401. The received signals for each channel may be processed by a single low-noise amplifier (LNA) 405 and a group of LNAs (e.g., 8-16 low-noise amplifiers) may share a single analog to digital converter (ADC) 409. In some embodiments, a symmetric cryptographic algorithm or encryption 411 may be implemented after or on the ADC 409 itself prior to output data 413 being generated.

In the example illustrated in FIG. 4A, the neural interface has a total of M×N channels, as each recording array 403 has a total of N channels 407, and each neural interface may have a total of M different recording arrays. For example, assuming the ADC 409 is a 10-bit ADC, the neural interface produces about 10×M bits of simultaneous data (e.g., M recording arrays each producing 10 bits of data from the ADC). This high number of parallel data allows for the use of R parallel stream or block ciphers (even 128- or 256-block ciphers) without the need for padding. For example, the R parallel stream or block ciphers can reach the required input-bits for the cipher without requiring the adding of additional bits. For example, the high number of parallel data may not require adding 28 bits to 10 ADCs with 10-bit resolution each in order to reach the required 128 bits for a 128-block cipher.

Similarly, as illustrated in FIG. 4B, a stimulating array 415 may also implement symmetric, hardware-friendly encryption algorithms. As illustrated the incoming data 425 including data for controlling the operation of the stimulator through the stimulator controller 421 may be encrypted, until it reaches a de-encryption block 423 which is configured to decrypt the data using a S parallel stream or block ciphers. Each neural interface may be composed of a plurality of stimulator arrays (P) 415, each of which is configured to stimulate across Q channels 417 via the electrode array 401. The Q channels may be multiplexed.

The disclosed system with neural stimulator array 415 may also utilize data parallelism when implementing de-encryption. For example, in the case of visual prosthesis applications, cryptographic algorithms may be used to provide data rates of 100s of Mb/s for thousands of channels.

FIGS. 5A-5J illustrate circuit-level techniques that enable the low-power, compact implementation of symmetric encryption and decryption algorithms on scalable, high-bandwidth neural interfaces. Thus, FIG. 5A-5J illustrate different techniques for implementing in-body, hardware-level encryption of neural data in the context of a high-bandwidth neural interface, including while using a successive-approximation register (SAR).

FIG. 5A illustrates a circuit block diagram of a successive-approximation register (SAR) ADC, as is commonly used in neural interfaces. The ADC block diagram includes input voltage 501, a sample and hold circuit 503, a comparator 505, a digital-to-analog converter 509, a binary search algorithm 507, and an output 513. Advantageously, the illustrated circuit in FIG. 5A may provide a simple efficient system with low complexity, that provides sufficient resolution with lower power requirements. The illustrated circuit may provide an adequate processing speed for use in neural interfaces.

FIG. 5B illustrates a circuit block diagram for a modified SAR ADC in accordance with some embodiments of the present disclosure, as is used in connection with a neural interface. In particular, FIG. 5B illustrates a modified SAR ADC which includes a multiplexer 511 that is configured to send the digitized bit one-by-one 515 to a 1-bit Stream Cypher 517 after every conversion cycle. Thus, the circuit 502 encrypts the data during digitization, and the data gets simultaneously digitized and encrypted, thus producing encrypted serial output 519. Although in the circuit illustrated in FIG. 5B, the data is encrypted right in the ADC 502, the nature of the standard SAR algorithm 507 produces an internal digital representation of the data at the input of the DAC 509, before the multiplexor 511 is applied that is not secured or unencrypted. The circuit illustrated in FIG. 5B may be easier to implement. However, the circuit of FIG. 5B still generates a digital representation of the data at the input of the DAC.

FIG. 5C illustrates a circuit block diagram for a second modified SAR ADC. To address the production of unencrypted data in the form of the standard SAR algorithm 507 producing an internal digital representation of the data at the input to DAC 509 (as was illustrated in FIG. 5B), the circuit 504 of FIG. 5C includes the presence of multiplexor 523 and an accumulator 525 at the input of the circuit, after the sample and hold circuit 503, and is configured to produce encrypted serial output 527. This modified configuration of the circuit 504 illustrated in FIG. 5C allows for implementing the binary search algorithm 507 such that the input of the DAC 509 is reset after every conversion cycle. In this way, the disclosed embodiment may have no memory of converted bits in the ADC 504 and the system never generates full digital words (group of bits) representing unencrypted neural data, thus providing enhanced security. The complex circuit block illustrated in FIG. 5C provides enhanced security in comparison to other embodiments.

FIG. 5D illustrates a circuit block diagram in accordance with embodiments of the present disclosure, where the serial outputs of eight (8) parallel modified ADCs 601 similar to those in the circuit of FIG. 5B, are grouped together to be the input of a 1-byte Stream Cipher 603 and produce encrypted output 605. As illustrated in connection with FIG. 5B, each of the eight (8) parallel modified ADCs 601 may include a circuit where the input 501 is received by a sample and hold circuit 503, a comparator 505, a digital-to-analog converter 509, a binary search algorithm 507, and then routed to a multiplexer 511 that is configured to send the digitized bit one-by-one 515 to the 1-byte stream cipher 603. In some embodiments, a scalable, high-bandwidth neural interface may include more than 8 ADCs. In such an embodiment, subsets of total number of ADCs may be grouped such that each group of 8 ADCs may be connected to a 1-byte stream cipher. Advantageously, in the embodiment illustrated in FIG. 5D, the encrypted data may be received from multiple ADCs which may provide an additional layer of security to the embodiment. However, the illustrated embodiment in FIG. 5D does generate a digital representation of the data at the input of the DAC.

FIG. 5E illustrates a circuit block diagram in accordance with embodiments of the present disclosure, where the serial outputs of eight (8) parallel modified ADCs 607 similar to those in the circuit of FIG. 5C, are grouped together to be the input of a 1-byte Stream Cipher 609 and produce encrypted output 611. As illustrated in connection with FIG. 5C, each of the eight (8) parallel modified ADCs 607 may include a circuit where the input 501 is received by a sample and hold circuit 503, a multiplexer 523 and accumulator 525, a comparator 505, a digital-to-analog converter 509, a binary search algorithm 507, and then routed to a multiplexer 515 that is configured to send the digitized bit one-by-one 515 to the 1-byte stream cipher 609 to produce encrypted output 611. In some embodiments, a scalable, high-bandwidth neural interface may include more than 8 ADCs. In such an embodiment, subsets of total number of ADCs may be grouped such that each group of 8 ADCs may be connected to a 1-byte stream cipher. While the embodiment described in FIG. 5E is more complex, it may provide the advantage of enhanced security.

FIG. 5F illustrates a circuit block diagram 613 where the serial outputs of 128 parallel modified ADCs 615 are grouped together to be the input of a 128-bit Block Cipher 617 to produce encrypted output 619. In some embodiments, the 128 parallel modified ADCs 615 may be ADCs such as those illustrated in FIG. 5B. Similar to the embodiment illustrated in FIG. 5F, 192 or 256 parallel SAR ADCs can be grouped together instead of the illustrated modified ADCs 615 and connect to 192- or 256-bit Block Ciphers instead of the 128-bit Block Cipher 617. In embodiments where the scalable, high-bandwidth neural interface has more ADCs, the ADCs may be grouped every 128-bit, 192-bit, or 256-bit such that each group gets connected to a 128-bit, 192-bit, or 256-bit Block Cipher, respectively. Advantageously, in the embodiment illustrated in FIG. 5F, the encrypted data may be received from multiple ADCs which may provide an additional layer of security to the embodiment.

FIG. 5G illustrates a circuit block diagram 621 where the serial outputs of modified ADCs 623 are grouped together to be the input of a block cipher 625 and produce encrypted output 627. In some embodiments, the modified ADCs 623 may include 128 parallel modified ADCs such as those illustrated in FIG. 5C. In some embodiments, the block cipher 625 may be a 128-bit Block Cipher. Similarly, 192-bit or 256-bit parallel SAR ADCs can be grouped together and connect to 192- or 256-bit Block Ciphers, respectively. In some embodiments, the scalable, high-bandwidth neural interface may include more ADCs, and all the ADCs may be grouped every 128, 192, or 256 such that each group gets connected to a 128-, 192-, or 256-bit Block Cipher. Advantageously, in the embodiment illustrated in FIG. 5G, the encrypted data may be received from multiple ADCs which may provide an additional layer of security to the embodiment.

FIG. 5H illustrates a circuit block diagram 621 where the serial outputs of “B” parallel modified ADCs 631, are grouped together to be the input of a “B”-bit, unrolled, pipelined Stream or Block Cipher 633 to produce encrypted output 635. In some embodiments the “B” parallel modified ADCs 631 may be similar to those illustrated in FIG. 5B. The circuit 621 illustrated in FIG. 5H may be configured to utilize the cyclic nature of the ADC to perform rounds of the cipher simultaneously with the binary search algorithm of the ADC. The illustrated circuit block diagram 621 includes a “B” that may be 1, 8, 128, 192, or 256 bits. In the case that the scalable, high-bandwidth neural interface has more ADCs, all the ADCs are grouped every 1, 8, 128, 192, or 256 such that each group gets connected to a 1-, 8-, 128-, 192-, or 256-bit, unrolled, pipelined cipher. Advantageously, the embodiment illustrated in FIG. 5H, may provide for increased encryption speeds.

FIG. SI illustrates a circuit block diagram 637 where the serial outputs of “B” parallel modified ADCs 639 are grouped together to be the input of a B-bit, unrolled, pipelined Stream or Block Cipher 641 configured to produce encrypted output 643. In some embodiments, the “B” parallel modified ADCs 639 may be similar to those illustrated in FIG. 5C. The circuit 637 illustrated in FIG. 5I may be configured to utilize the cyclic nature of the ADC to perform rounds of the cipher simultaneously with the binary search algorithm of the ADC. The illustrated circuit block diagram 621 includes a “B” that may be 1, 8, 128, 192, or 256 bits. In the case that the scalable, high-bandwidth neural interface has more ADCs, all the ADCs are grouped every 1, 8, 128, 192, or 256 such that each group gets connected to a 1-, 8-, 128-, 192-, or 256-bit, unrolled, pipelined Cipher.

Embodiments of the present disclosure may include one or more encryption algorithms. Encryption algorithms may include a set of mathematical operations that may be repeated a specific number of times to the input data, in successive fashion such that the output of each set of operations will become the input of the next one. For example, a Sa1sa20 algorithm may be configured to apply the specific set of operations to the data (and its subsequent outputs) a total of 20 times. In this manner, the algorithm can be applied in a loop (i.e., sequentially) or it can be in an open loop (i.e., unrolled), where it is applied in a more parallel fashion.

FIG. 5J illustrates a system block diagram of an implementation of data encryption for neural recording applications where encryption is done following feature extraction from the neural data. As illustrated the system 711 may include an electrode array 709 configured to send signals to a recording array 701. The signals from the recording array 701 may be transmitted to a processor 703 that is configured to apply techniques such as digital signal processing (DSP), feature extraction and compression to the raw data. The processed data may then undergo encryption 705 and the resulting data 707 may be encrypted. The system disclosed in FIG. 5J provides the ability to signal process on the raw data stream which provides advantages in terms of compression, power savings, and the like. However, as encryption is done after the processing on the raw data, this may pose a security risk.

FIG. 5A-5J illustrate various techniques for implementing in-body, hardware-level encryption of neural data in the context of a high-bandwidth neural interface, including while using a successive-approximation register (SAR). The disclosed encryption techniques may take place within the body, and more specifically, within the skull.

In some embodiments, the encryption may be performed during digitization by the analog-to-digital converter, thus the system is configured such that it never generates digital bits representing un-encrypted neural data.

In some embodiments, the encryption may be performed following feature extraction from the neural data, thus users will not lose the ability to perform signal processing on the raw data stream which may provide compression and power savings benefits.

In some embodiments, the encryption may be performed during digitization. In such an embodiment the system may never generate a full digital word (group of bits) representing un-encrypted neural data.

As illustrated above, in some embodiments, the serial output of 1, 8, 128, 192, 256 parallel, modified ADCs may be grouped together to form the input of a 1-, 8-, 128-, 192-, 256-bit symmetric stream or block ciphers. The data may then be encrypted following the respective symmetric cryptographic algorithm.

In some embodiments, the serial output of 1, 8, 128, 192, 256 parallel, modified ADCs may be grouped together to form the input of a 1-, 8-, 128-, 192-, 256-bit, unrolled, pipelined stream or block ciphers. In such an embodiment, the cyclic nature of the ADC may be advantaged to perform rounds of the cipher simultaneously with the binary search algorithm of the ADC. The data may then be encrypted following the respective symmetric cryptographic algorithm.

Alternatively, in some embodiments the data encryption may be performed following feature extraction from the neural data. The advantage of such a system would be not losing the ability to perform signal processing on the raw data stream for compression, power savings, and the like.

Accordingly, the disclosed systems and methods may be utilized for front-end integrated circuits in high-bandwidth neural interfaces. As high-bandwidth neural interfaces include integrated circuits that are typically used to communicate via a wire with another electronic chips within the same device for wireless communication the security issues associated with the integrated circuits may be addressed via data encryption. Other security aspects such as authentication may be implemented in other ICs of the neural interface.

The disclosed systems and methods provide encryption systems for neural interfaces, where the encryption may be performed in body, on the head, at the point of data generation, or as part of the architecture of the neural interfaces. Additionally, or alternatively, data encryption may be conducted outside of the body. The disclosed methods and system may include both hardware and software components.

1. A neural interface device comprising:
an electrode array configured to stimulate or record from neural tissue adjacent to the electrode array; and
an integrated circuit in electrical communication with the electrode array, the integrated circuit comprising an analog-to-digital converter (ADC) producing digitized electrical signal output, wherein the ADC comprises an encryption module, wherein the encryption module encrypts the digitized electrical signal output of the ADC.

2. The neural interface device of claim 1, wherein the ADC comprises a successive approximation register (SAR) architecture.

3. The neural interface device of claim 1, wherein the encryption module comprises a bit stream cipher, wherein the encryption module applies the bit stream cipher to the digitized electrical signal output of the ADC.

4. The neural interface device of claim 1, wherein the encryption module comprises a block stream cipher, wherein the encryption module applies the block stream cipher to the digitized electrical signal output of the ADC.

5. The neural interface device of claim 1, wherein a cipher of the encryption module comprises 1-, 8-, 128-, 192- or 256-bits.

6. The neural interface device of claim 1, comprising:
a wireless transmitter communicatively coupled to the integrated circuit or the encryption module and an external processor.

7. The neural interface device of claim 1, comprising:
control logic for operating the integrated circuit or electrode array;
memory for storing recordings from the electrode array; and
a power management unit for providing power to the integrated circuit or electrode array.

8. A neural interface device comprising:
an electrode array configured to stimulate or record from neural tissue adjacent the electrode array; and
an integrated circuit in electrical communication with the electrode array, the integrated circuit comprising a successive approximation register (SAR) analog-to-digital converter (ADC), wherein the SAR ADC comprises a cipher module, wherein the cipher module applies a cipher to an electrical signal output by a digital-to-analog circuit (DAC) of the SAR ADC to generate an encrypted serial output.

9. The neural interface device of claim 8, wherein the cipher comprises a bit stream cipher or a block cipher.

10. The neural interface device of claim 8, wherein the SAR ADC comprises:
a sample and hold circuit configured to receive the electrical signal from the electrode array;
a comparator electrically coupled to the sample and hold circuit, wherein the comparator compares the received electrical signal to a reference signal of the DAC, wherein the DAC is electrically coupled to the comparator; and
a binary search algorithm electrically coupled to the comparator, wherein the binary search algorithm generates a digital electrical signal representative of the received electrical signal.

11. The neural interface device of claim 8, wherein the cipher module comprises at least one of 1-, 8-, 128-, 192-, or 256-bits.

12. The neural interface device of claim 8, comprising:
a wireless transmitter communicatively coupled to the integrated circuit or a cipher block module, and an external processor.

13. The neural interface device of claim 8, comprising:
control logic for operating the integrated circuit or electrode array;
memory for storing recordings from the electrode array; and
a power management unit for providing power to the integrated circuit or electrode array.

14. A neural interface device comprising:
an electrode array configured to stimulate or record from neural tissue adjacent the electrode array;
at least one recording array, each of the at least one recording array having at least one integrated circuit in electrical communication with the electrode array, wherein each of the at least one integrated circuits comprises a successive approximation register (SAR) analog-to-digital converter (ADC), wherein the SAR ADC receives an electrical signal from the at least one recording array and outputs a digital electrical signal; and
a stream cipher module comprising a symmetric cryptographic algorithm, wherein the stream cipher module applies the symmetric cryptographic algorithm to the digital electrical signal output by the SAR ADC to generate serial encrypted output.

15. The neural interface device of claim 14, wherein the SAR ADC comprises:
a sample and hold circuit configured to receive the electrical signal from the at least one recording array;
a comparator electrically coupled to the sample and hold circuit, wherein the comparator compares the received electrical signal to a reference signal of a digital-to-analog circuit (DAC) of the SAR ADC, wherein the DAC is electrically coupled to the comparator; and
a binary search algorithm electrically coupled to the comparator, wherein the binary search algorithm generates the digital electrical signal representative of the received electrical signal.

16. A neural interface device comprising:
an electrode array configured to stimulate or record from neural tissue adjacent the electrode array;
a plurality of recording arrays each having at least one integrated circuit in electrical communication with the electrode array, wherein each of the at least one integrated circuits comprises a successive approximation register (SAR) analog-to-digital converter (ADC), wherein the SAR ADC receives an electrical signal from the plurality of recording arrays and outputs a digital electrical signal; and
a block cipher module comprising a symmetric cryptographic algorithm, wherein the block cipher module applies the symmetric cryptographic algorithm to the digital electrical signal output by the SAR ADC to generate serial encrypted output.

17. The neural interface device of claim 16, wherein the SAR ADC comprises:
a sample and hold circuit configured to receive the electrical signal from the at least one recording array;
a comparator electrically coupled to the sample and hold circuit, wherein the comparator compares the received electrical signal to a reference signal of a digital-to-analog circuit (DAC) of the SAR ADC, wherein the DAC is electrically coupled to the comparator; and
a binary search algorithm electrically coupled to the comparator, wherein the binary search algorithm generates the digital electrical signal representative of the received electrical signal.

18. A neural interface device comprising:
an electrode array configured to stimulate or record from neural tissue adjacent the electrode array;
a plurality of recording arrays each having at least one integrated circuit in electrical communication with the electrode array, wherein each of the at least one integrated circuits comprises a successive approximation register (SAR) analog-to-digital converter (ADC), wherein the SAR ADC receives an electrical signal from the plurality of recording arrays and outputs a digital electrical signal; and
an unrolled pipelined cipher module comprising a symmetric cryptographic algorithm, wherein the unrolled pipelined cipher module applies the symmetric cryptographic algorithm to the digital electrical signal output by the SAR ADC to generate serial encrypted output.

19. The neural interface device of claim 18, wherein the SAR ADC comprises:
a sample and hold circuit configured to receive the electrical signal from at least one recording array;
a comparator electrically coupled to the sample and hold circuit, wherein the comparator compares the received electrical signal to a reference signal of a digital-to-analog circuit (DAC) of the SAR ADC, wherein the DAC is electrically coupled to the comparator; and
a binary search algorithm electrically coupled to the comparator, wherein the binary search algorithm generates the digital electrical signal representative of the received electrical signal.
